(19) United States
(12) Patent Application Publication
(10) Pub. No.: US 2009/0163452 A1
(43) Pub. Date: Jun. 25, 2009

(54) COMPOSITIONS AND METHODS FOR LOWERING SERUM CHOLESTEROL

(76) Inventor: Janice B. SCHWARTZ, San Francisco, CA (US)

Correspondence Address: MORRISON & FOERSTER LLP, 755 PAGE MILL RD, PALO ALTO, CA, 94304-1018, US

(21) Appl. No.: 12/274,189

(22) Filed: Nov. 19, 2008

Related U.S. Application Data
(60) Provisional application No. 61/088,320, filed on Aug. 12, 2008.
(60) Provisional application No. 61/008,552, filed on Dec. 20, 2007.

(51) Int. Cl. A61K 31/59

(52) U.S. Cl. 514/167

(57) ABSTRACT

The invention provides a method for lowering circulating LDL-cholesterol or total cholesterol in a human in need thereof, comprising: orally administering to the human a therapeutically effective amount of a statin and vitamin D daily for at least about 6 weeks, wherein the vitamin D is administered by one or more pharmaceutical compositions.

### CROSS-REFERENCE TO RELATED APPLICATIONS

This application claims the benefit under 35 U.S.C. §119(e) of U.S. Provisional Applications Ser. No. 61/008,552, filed Dec. 20, 2007, and 61/088,320, filed Aug. 12, 2008, the contents of which are incorporated herein by reference in their entirety.

### STATEMENT REGARDING FEDERALLY SPONSORED RESEARCH OR DEVELOPMENT

This study was funded in part by grants R01 AG 15982, National Institutes of Health, in part with resources of the Jewish Home of San Francisco, Calif., and in part by Grant Number UL1 RR024131 from the National Center for Research Resources (NCRR), a component of the National Institutes of Health (NIH), and NIH Roadmap for Medical Research. The Federal Government may have certain rights in this invention.

### BACKGROUND OF THE INVENTION

Cardiovascular disease is the leading cause of death of men and women in the United States. Treatment and prevention of cardiovascular disease often involves strategies to reduce cholesterol concentrations. In western civilizations, cholesterol concentrations rise with age in people. Therapeutic lifestyle modifications including dietary restriction and exercise and weight loss in the presence of obesity are recommended as the first line approach. However, dietary intake accounts for only about 15% of daily cholesterol production in the body, and complete elimination of cholesterol containing foods is seldom possible. Additionally, even successful weight loss regimens have high recidivism rates, and a sedentary lifestyle appears to be the norm in western civilizations. Thus, lifestyle modification is not achieved in most patients, and for the patient able to modify lifestyle with moderate dietary restriction and exercise, they are only able to lower cholesterol and LDL-cholesterol concentrations on the order of 10-15%.

Lipid lowering medications have become the most commonly prescribed drugs in the United States, with atorvastatin the single most commonly prescribed HMG CoA reductase inhibitor in the United States and worldwide. (Loftus P. Pfizer's Lipitor Patent Reissue Rejected. The Wall Street Journal Online. 2007 Aug. 16.) HMG CoA reductase inhibitors (statins) have a loglinear dose response and doubling of doses results in only an average 7-10% additional cholesterol reduction. Statins can have side effects and have significant costs. Statins can lead to hepatic toxicity that may be dose-related, and repeated testing of liver enzyme levels are recommended throughout their chronic administration. Severe muscle damage (rhabdomyolysis) is less common, but can lead to renal failure and death. Muscle weakness and interference with the ability to perform daily functions may occur in over 10% of older people receiving statins and require cessation. Interactions with other medications can occur with statins metabolized by the most common hepatic and intestinal cytochrome P450(CYP) enzyme, CYP3A. Cholesterol absorption inhibitors, which may be administered in conjunction with statins, have significant gastrointestinal side effects and may be considered intolerable by patients. All are currently prescription drugs and entail considerable monetary cost because of the need for chronic administration and monitoring.

In humans, atorvastatin has low oral bioavailability chiefly due to extensive first-pass metabolism, both in the intestine and in the liver by CYP3A. (Lennernas H. Clinical pharmacokinetics of atorvastatin. Clin Pharmacokinet. 2003;42:1141-60.) Atorvastatin is administered as atorvastatin acid that is extensively metabolized via lactonization and CYP3A4-mediated oxidations to form two major active metabolites, 2-hydroxy-atorvastatin acid and 4-hydroxyatorvastatin acid. These hydroxylated metabolites are excreted into bile in unchanged form or as glucuronide conjugates. (Jacobsen W, et al. Lactonization is the critical first step in the disposition of the 3-hydroxy-3-methylglutaryl-CoA reductase inhibitor atorvastatin. Drug Metab Dispos. 2000;28:1369-78).

All patents, patent applications, documents, and articles cited herein are herein incorporated by reference in their entirety.

### BRIEF SUMMARY OF THE INVENTION

In one aspect of the invention is a method for lowering circulating LDL-cholesterol or total cholesterol in a human in need thereof, comprising: orally administering to the human a therapeutically effective amount of a statin and vitamin D daily for at least about 6 weeks, wherein the vitamin D is administered by one or more pharmaceutical compositions. In some embodiments, the human is a male or a pre-menopausal female. In some embodiments, the human is male. In some embodiments, the human is a pre-menopausal female. In some embodiments, the human has congestive heart failure. In some embodiments, the human does not have congestive heart failure. In some embodiments, the total daily amount of vitamin D administered by the one or more pharmaceutical compositions is equipotent to about 600 to about 2500 IU of ergocalciferol or cholechalciferol. In some embodiments, the total daily amount of vitamin D administered by the one or more pharmaceutical compositions is equipotent to about 1200 to about 2500 IU of ergocalciferol or cholechalciferol. In some embodiments, the total daily amount of vitamin D administered by the one or more pharmaceutical compositions is equipotent to about 600 to about 2000 IU of ergocalciferol or cholechalciferol. In some embodiments, the total daily amount of vitamin D administered by the one or more pharmaceutical compositions is equipotent to about 600 to about 1800 IU of ergocalciferol or cholechalciferol. In some embodiments, the total daily amount of vitamin D administered by the one or more pharmaceutical compositions is equipotent to about 600 to about 1500 IU of ergocalciferol or cholechalciferol. In some embodiments, the total daily amount of vitamin D administered by the one or more pharmaceutical compositions is equipotent to about 600 to about 1200 IU of ergocalciferol or cholechalciferol. In some embodiments, the total daily amount of vitamin D administered by the one or more pharmaceutical compositions is equipotent to about 600 to about 1000 IU of ergocalciferol or cholechalciferol. In some embodiments, the total daily amount of vitamin D administered by the one or more pharmaceutical compositions is equipotent to about 600 to about 800 IU of ergocalciferol or cholechalciferol. In some embodiments, the total daily amount of vitamin D administered by the one or more pharmaceutical compositions is equipotent to about 800 to about 1000 IU of ergocalciferol or cholechalciferol. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin. In some embodiments, the amount of statin required to achieve a particular decrease in LDL-cholesterol or total cholesterol is less than that required when the statin is administered in the absence of the one or more pharmaceutical compositions comprising vitamin D. In some embodiments, the statin and the vitamin D are administered daily for at least about 8 weeks. In some embodiments, the statin and the vitamin D are administered daily for at least about 12 weeks. In some embodiments, the statin and the vitamin D are administered daily for at least about 4 months. In some embodiments, the statin and the vitamin D are administered daily for at least about 6 months. In some embodiments, the vitamin D is administered in a single daily dose. In some embodiments, the vitamin D is administered in two or more daily doses. In some embodiments, the statin is administered in a single daily dose. In some embodiments, the statin is administered in two or more daily doses. In some embodiments, the method further comprises orally administering to the human calcium daily for at least about 6 weeks. In some embodiments, the total daily amount of calcium administered is at least about 300 mg. In some embodiments, the total daily amount of calcium administered is at least about 500 mg. In some embodiments, the total daily amount of calcium administered is about 500 to about 2000 mg. In some embodiments, the calcium comprises one or more of: calcium carbonate, calcium phosphate, and calcium citrate. In some embodiments, the method further comprises administering one or more additional therapeutic agents selected from the group consisting of: a non-statin lipid lowering agent, a HDL-raising agent, insulin, and a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, the one or more additional therapeutic agents comprise ezetimide. In some embodiments, the one or more pharmaceutical compositions comprises a pharmaceutical composition comprising vitamin D and calcium. In some embodiments, the one or more pharmaceutical compositions comprises a pharmaceutical composition comprising a multivitamin composition comprising vitamin D. In some embodiments, the one or more pharmaceutical compositions comprises a pharmaceutical composition comprising the statin and vitamin D. In some embodiments, the one or more pharmaceutical compositions comprises a pharmaceutical composition comprising the statin, vitamin D, and calcium. In some embodiments, the statin is administered in a separate dosage form from the one or more pharmaceutical compositions comprising vitamin D. In some embodiments, the human is at least about 12 years of age. In some embodiments, the human is at least about 20 years of age. In some embodiments, the human is at least about 30 years of age. In some embodiments, the human does not have psoriasis. In some embodiments, the human does not have osteoporosis. In some embodiments, the human does not have multiple sclerosis. In some embodiments, the human is diabetic. In some embodiments, the human has not previously been treated with at least about 600 IU of ergocalciferol or cholecalciferol or equipotent amount thereof per day for at least about 6 weeks. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, the circulating LDL-cholesterol or total cholesterol levels are serum levels. In some embodiments, the circulating LDL-cholesterol or total cholesterol levels are plasma levels.

In another aspect of the invention is a method for lowering circulating LDL-cholesterol or total cholesterol in a human in need thereof, comprising: orally administering to the human a therapeutically effective amount of a statin and vitamin D daily for at least about 6 weeks, wherein the human is unable to tolerate the statin when administered in a therapeutically effective dose and in the absence of vitamin D; and wherein the vitamin D is administered by one or more pharmaceutical compositions. In some embodiments, the human is male. In some embodiments, the human is a pre-menopausal female. In some embodiments, the human has congestive heart failure. In some embodiments, the human does not have congestive heart failure. In some embodiments, the total daily amount of vitamin D administered by the one or more pharmaceutical compositions is equipotent to about 600 to about 2500 IU of ergocalciferol or cholechalciferol. In some embodiments, the total daily amount of vitamin D administered by the one or more pharmaceutical compositions is equipotent to about 1200 to about 2500 IU of ergocalciferol or cholechalciferol. In some embodiments, the total daily amount of vitamin D administered by the one or more pharmaceutical compositions is equipotent to about 600 to about 2000 IU of ergocalciferol or cholechalciferol. In some embodiments, the total daily amount of vitamin D administered by the one or more pharmaceutical compositions is equipotent to about 600 to about 1800 IU of ergocalciferol or cholechalciferol. In some embodiments, the total daily amount of vitamin D administered by the one or more pharmaceutical compositions is equipotent to about 600 to about 1500 IU of ergocalciferol or cholechalciferol. In some embodiments, the total daily amount of vitamin D administered by the one or more pharmaceutical compositions is equipotent to about 600 to about 1200 IU of ergocalciferol or cholechalciferol. In some embodiments, the total daily amount of vitamin D administered by the one or more pharmaceutical compositions is equipotent to about 600 to about 1000 IU of ergocalciferol or cholechalciferol. In some embodiments, the total daily amount of vitamin D administered by the one or more pharmaceutical compositions is equipotent to about 600 to about 800 IU of ergocalciferol or cholechalciferol. In some embodiments, the total daily amount of vitamin D administered by the one or more pharmaceutical compositions is equipotent to about 800 to about 1000 IU of ergocalciferol or cholechalciferol. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin. In some embodiments, the amount of statin required to achieve a particular decrease in LDL-cholesterol or total cholesterol is less than that required when the statin is administered in the absence of the one or more pharmaceutical compositions comprising vitamin D. In some embodiments, the statin and the vitamin D are administered daily for at least about 8 weeks. In some embodiments, the statin and the vitamin D are administered daily for at least about 12 weeks. In some embodiments, the statin and the vitamin D are administered daily for at least about 4 months. In some embodiments, the statin and the vitamin D are administered daily for at least about 6 months. In some embodiments, the vitamin D is administered in a single daily dose. In some embodiments, the vitamin D is administered in two or more daily doses. In some embodiments, the statin is administered in a single daily dose. In some embodiments, the statin is administered in two or more daily doses. In some embodiments, the method further comprises orally administering to the human calcium daily for at least about 6 weeks. In some embodiments, the total daily amount of calcium administered is at least about 300 mg. In some embodiments, the total daily amount of calcium administered is at least about 500 mg. In some embodiments, the total daily amount of calcium administered is about 500 to about 2000 mg. In some embodiments, the calcium comprises one or more of: calcium carbonate, calcium phosphate, and calcium citrate. In some embodiments, the method further comprises administering one or more additional therapeutic agents selected from the group consisting of: a non-statin lipid lowering agent, a HDL-raising agent, insulin, and a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, the one or more additional therapeutic agents comprise ezetimide. In some embodiments, the one or more pharmaceutical compositions comprises a pharmaceutical composition comprising vitamin D and calcium. In some embodiments, the one or more pharmaceutical compositions comprises a pharmaceutical composition comprising a multivitamin composition comprising vitamin D. In some embodiments, the one or more pharmaceutical compositions comprises a pharmaceutical composition comprising the statin and vitamin D. In some embodiments, the one or more pharmaceutical compositions comprises a pharmaceutical composition comprising the statin, vitamin D, and calcium. In some embodiments, the statin is administered in a separate dosage form from the one or more pharmaceutical compositions comprising vitamin D. In some embodiments, the human is at least about 12 years of age. In some embodiments, the human is at least about 20 years of age. In some embodiments, the human is at least about 30 years of age. In some embodiments, the human does not have psoriasis. In some embodiments, the human does not have osteoporosis. In some embodiments, the human does not have multiple sclerosis. In some embodiments, the human is diabetic. In some embodiments, the human has not previously been treated with at least about 600 IU of ergocalciferol or cholecalciferol or equipotent amount thereof per day for at least about 6 weeks. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, the circulating LDL-cholesterol or total cholesterol levels are serum levels. In some embodiments, the circulating LDL-cholesterol or total cholesterol levels are plasma levels.

In another aspect of the invention is a method for lowering circulating LDL-cholesterol or total cholesterol in a human in need thereof, comprising: orally administering to the human a therapeutically effective amount of a statin and vitamin D daily for at least about 6 weeks, wherein the human is a pre-menopausal female; and wherein the vitamin D is administered by one or more pharmaceutical compositions. In some embodiments, the human has congestive heart failure. In some embodiments, the human does not have congestive heart failure. In some embodiments, the total daily amount of vitamin D administered by the one or more pharmaceutical compositions is equipotent to about 600 to about 2500 IU of ergocalciferol or cholechalciferol. In some embodiments, the total daily amount of vitamin D administered by the one or more pharmaceutical compositions is equipotent to about 1200 to about 2500 IU of ergocalciferol or cholechalciferol. In some embodiments, the total daily amount of vitamin D administered by the one or more pharmaceutical compositions is equipotent to about 600 to about 2000 IU of ergocalciferol or cholechalciferol. In some embodiments, the total daily amount of vitamin D administered by the one or more pharmaceutical compositions is equipotent to about 600 to about 1800 IU of ergocalciferol or cholechalciferol. In some embodiments, the total daily amount of vitamin D administered by the one or more pharmaceutical compositions is equipotent to about 600 to about 1500 IU of ergocalciferol or cholechalciferol. In some embodiments, the total daily amount of vitamin D administered by the one or more pharmaceutical compositions is equipotent to about 600 to about 1200 IU of ergocalciferol or cholechalciferol. In some embodiments, the total daily amount of vitamin D administered by the one or more pharmaceutical compositions is equipotent to about 600 to about 1000 IU of ergocalciferol or cholechalciferol. In some embodiments, the total daily amount of vitamin D administered by the one or more pharmaceutical compositions is equipotent to about 600 to about 800 IU of ergocalciferol or cholechalciferol. In some embodiments, the total daily amount of vitamin D administered by the one or more pharmaceutical compositions is equipotent to about 800 to about 1000 IU of ergocalciferol or cholechalciferol. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin. In some embodiments, the amount of statin required to achieve a particular decrease in LDL-cholesterol or total cholesterol is less than that required when the statin is administered in the absence of the one or more pharmaceutical compositions comprising vitamin D. In some embodiments, the statin and the vitamin D are administered daily for at least about 8 weeks. In some embodiments, the statin and the vitamin D are administered daily for at least about 12 weeks. In some embodiments, the statin and the vitamin D are administered daily for at least about 4 months. In some embodiments, the statin and the vitamin D are administered daily for at least about 6 months. In some embodiments, the vitamin D is administered in a single daily dose. In some embodiments, the vitamin D is administered in two or more daily doses. In some embodiments, the statin is administered in a single daily dose. In some embodiments, the statin is administered in two or more daily doses. In some embodiments, the method further comprises orally administering to the human calcium daily for at least about 6 weeks. In some embodiments, the total daily amount of calcium administered is at least about 300 mg. In some embodiments, the total daily amount of calcium administered is at least about 500 mg. In some embodiments, the total daily amount of calcium administered is about 500 to about 2000 mg. In some embodiments, the calcium comprises one or more of: calcium carbonate, calcium phosphate, and calcium citrate. In some embodiments, the method further comprises administering one or more additional therapeutic agents selected from the group consisting of: a non-statin lipid lowering agent, a HDL-raising agent, insulin, and a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, the one or more additional therapeutic agents comprise ezetimide. In some embodiments, the one or more pharmaceutical compositions comprises a pharmaceutical composition comprising vitamin D and calcium. In some embodiments, the one or more pharmaceutical compositions comprises a pharmaceutical composition comprising a multivitamin composition comprising vitamin D. In some embodiments, the one or more pharmaceutical compositions comprises a pharmaceutical composition comprising the statin and vitamin D. In some embodiments, the one or more pharmaceutical compositions comprises a pharmaceutical composition comprising the statin, vitamin D, and calcium. In some embodiments, the statin is administered in a separate dosage form from the one or more pharmaceutical compositions comprising vitamin D. In some embodiments, the human is at least about 12 years of age. In some embodiments, the human is at least about 20 years of age. In some embodiments, the human is at least about 30 years of age. In some embodiments, the human does not have psoriasis. In some embodiments, the human does not have osteoporosis. In some embodiments, the human does not have multiple sclerosis. In some embodiments, the human is diabetic. In some embodiments, the human has not previously been treated with at least about 600 IU of ergocalciferol or cholecalciferol or equipotent amount thereof per day for at least about 6 weeks. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, the circulating LDL-cholesterol or total cholesterol levels are serum levels. In some embodiments, the circulating LDL-cholesterol or total cholesterol levels are plasma levels.

In another aspect of the invention is a method for lowering circulating LDL-cholesterol or total cholesterol in a human in need thereof, comprising: orally administering to the human a therapeutically effective amount of a statin and vitamin D daily for at least about 6 weeks, wherein the human is a male without congestive heart failure; and wherein the vitamin D is administered by one or more pharmaceutical compositions. In some embodiments, the total daily amount of vitamin D administered by the one or more pharmaceutical compositions is equipotent to about 600 to about 2500 IU of ergocalciferol or cholechalciferol. In some embodiments, the total daily amount of vitamin D administered by the one or more pharmaceutical compositions is equipotent to about 1200 to about 2500 IU of ergocalciferol or cholechalciferol. In some embodiments, the total daily amount of vitamin D administered by the one or more pharmaceutical compositions is equipotent to about 600 to about 2000 IU of ergocalciferol or cholechalciferol. In some embodiments, the total daily amount of vitamin D administered by the one or more pharmaceutical compositions is equipotent to about 600 to about 1800 IU of ergocalciferol or cholechalciferol. In some embodiments, the total daily amount of vitamin D administered by the one or more pharmaceutical compositions is equipotent to about 600 to about 1500 IU of ergocalciferol or cholechalciferol. In some embodiments, the total daily amount of vitamin D administered by the one or more pharmaceutical compositions is equipotent to about 600 to about 1200 IU of ergocalciferol or cholechalciferol. In some embodiments, the total daily amount of vitamin D administered by the one or more pharmaceutical compositions is equipotent to about 600 to about 1000 IU of ergocalciferol or cholechalciferol. In some embodiments, the total daily amount of vitamin D administered by the one or more pharmaceutical compositions is equipotent to about 600 to about 800 IU of ergocalciferol or cholechalciferol. In some embodiments, the total daily amount of vitamin D administered by the one or more pharmaceutical compositions is equipotent to about 800 to about 1000 IU of ergocalciferol or cholechalciferol. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin. In some embodiments, the amount of statin required to achieve a particular decrease in LDL-cholesterol or total cholesterol is less than that required when the statin is administered in the absence of the one or more pharmaceutical compositions comprising vitamin D. In some embodiments, the statin and the vitamin D are administered daily for at least about 8 weeks. In some embodiments, the statin and the vitamin D are administered daily for at least about 12 weeks. In some embodiments, the statin and the vitamin D are administered daily for at least about 4 months. In some embodiments, the statin and the vitamin D are administered daily for at least about 6 months. In some embodiments, the vitamin D is administered in a single daily dose. In some embodiments, the vitamin D is administered in two or more daily doses. In some embodiments, the statin is administered in a single daily dose. In some embodiments, the statin is administered in two or more daily doses. In some embodiments, the method further comprises orally administering to the human calcium daily for at least about 6 weeks. In some embodiments, the total daily amount of calcium administered is at least about 300 mg. In some embodiments, the total daily amount of calcium administered is at least about 500 mg. In some embodiments, the total daily amount of calcium administered is about 500 to about 2000 mg. In some embodiments, the calcium comprises one or more of: calcium carbonate, calcium phosphate, and calcium citrate. In some embodiments, the method further comprises administering one or more additional therapeutic agents selected from the group consisting of: a non-statin lipid lowering agent, a HDL-raising agent, insulin, and a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, the one or more additional therapeutic agents comprise ezetimide. In some embodiments, the one or more pharmaceutical compositions comprises a pharmaceutical composition comprising vitamin D and calcium. In some embodiments, the one or more pharmaceutical compositions comprises a pharmaceutical composition comprising a multivitamin composition comprising vitamin D. In some embodiments, the one or more pharmaceutical compositions comprises a pharmaceutical composition comprising the statin and vitamin D. In some embodiments, the one or more pharmaceutical compositions comprises a pharmaceutical composition comprising the statin, vitamin D, and calcium. In some embodiments, the statin is administered in a separate dosage form from the one or more pharmaceutical compositions comprising vitamin D. In some embodiments, the human is at least about 12 years of age. In some embodiments, the human is at least about 20 years of age. In some embodiments, the human is at least about 30 years of age. In some embodiments, the human does not have psoriasis. In some embodiments, the human does not have osteoporosis. In some embodiments, the human does not have multiple sclerosis. In some embodiments, the human is diabetic. In some embodiments, the human has not previously been treated with at least about 600 IU of ergocalciferol or cholecalciferol or equipotent amount thereof per day for at least about 6 weeks. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, the circulating LDL-cholesterol or total cholesterol levels are serum levels. In some embodiments, the circulating LDL-cholesterol or total cholesterol levels are plasma levels.

In another aspect of the invention is a method for lowering circulating LDL-cholesterol or total cholesterol in a human in need thereof, comprising: orally administering to the human a therapeutically effective amount of a statin and vitamin D daily for at least about 6 weeks, wherein the human is a male with congestive heart failure; wherein the vitamin D is administered by one or more pharmaceutical compositions; and wherein the total daily amount of vitamin D administered by the one or more pharmaceutical compositions is equipotent to about 600 to about 1800 International Units (IU). In some embodiments, the total daily amount of vitamin D administered by the one or more pharmaceutical compositions is equipotent to about 2200 to about 2500 IU of ergocalciferol or cholechalciferol. In some embodiments, the total daily amount of vitamin D administered by the one or more pharmaceutical compositions is equipotent to about 600 to about 1500 IU of ergocalciferol or cholechalciferol. In some embodiments, the total daily amount of vitamin D administered by the one or more pharmaceutical compositions is equipotent to about 600 to about 1200 IU of ergocalciferol or cholechalciferol. In some embodiments, the total daily amount of vitamin D administered by the one or more pharmaceutical compositions is equipotent to about 600 to about 1000 IU of ergocalciferol or cholechalciferol. In some embodiments, the total daily amount of vitamin D administered by the one or more pharmaceutical compositions is equipotent to about 600 to about 800 IU of ergocalciferol or cholechalciferol. In some embodiments, the total daily amount of vitamin D administered by the one or more pharmaceutical compositions is equipotent to about 800 to about 1000 IU of ergocalciferol or cholechalciferol. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin. In some embodiments, the amount of statin required to achieve a particular decrease in LDL-cholesterol or total cholesterol is less than that required when the statin is administered in the absence of the one or more pharmaceutical compositions comprising vitamin D. In some embodiments, the statin and the vitamin D are administered daily for at least about 8 weeks. In some embodiments, the statin and the vitamin D are administered daily for at least about 12 weeks. In some embodiments, the statin and the vitamin D are administered daily for at least about 4 months. In some embodiments, the statin and the vitamin D are administered daily for at least about 6 months. In some embodiments, the vitamin D is administered in a single daily dose. In some embodiments, the vitamin D is administered in two or more daily doses. In some embodiments, the statin is administered in a single daily dose. In some embodiments, the statin is administered in two or more daily doses. In some embodiments, the method further comprises orally administering to the human calcium daily for at least about 6 weeks. In some embodiments, the total daily amount of calcium administered is at least about 300 mg. In some embodiments, the total daily amount of calcium administered is at least about 500 mg. In some embodiments, the total daily amount of calcium administered is about 500 to about 2000 mg. In some embodiments, the calcium comprises one or more of: calcium carbonate, calcium phosphate, and calcium citrate. In some embodiments, the method further comprises administering one or more additional therapeutic agents selected from the group consisting of: a non-statin lipid lowering agent, a HDL-raising agent, insulin, and a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, the one or more additional therapeutic agents comprise ezetimide. In some embodiments, the one or more pharmaceutical compositions comprises a pharmaceutical composition comprising vitamin D and calcium. In some embodiments, the one or more pharmaceutical compositions comprises a pharmaceutical composition comprising a multivitamin composition comprising vitamin D. In some embodiments, the one or more pharmaceutical compositions comprises a pharmaceutical composition comprising the statin and vitamin D. In some embodiments, the one or more pharmaceutical compositions comprises a pharmaceutical composition comprising the statin, vitamin D, and calcium. In some embodiments, the statin is administered in a separate dosage form from the one or more pharmaceutical compositions comprising vitamin D. In some embodiments, the human is at least about 12 years of age. In some embodiments, the human is at least about 20 years of age. In some embodiments, the human is at least about 30 years of age. In some embodiments, the human does not have psoriasis. In some embodiments, the human does not have osteoporosis. In some embodiments, the human does not have multiple sclerosis. In some embodiments, the human is diabetic. In some embodiments, the human has not previously been treated with at least about 600 IU of ergocalciferol or cholecalciferol or equipotent amount thereof per day for at least about 6 weeks. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, the circulating LDL-cholesterol or total cholesterol levels are serum levels. In some embodiments, the circulating LDL-cholesterol or total cholesterol levels are plasma levels.

In another aspect of the invention is a kit consisting essentially of: (a) one or more unit doses of a statin; (b) one or more unit doses of vitamin D; (c) optionally one or more unit doses of calcium; and (d) instructions for use. In some embodiments, the kit comprises a single dosage form, wherein a unit dose of the statin and a unit dose of vitamin D are contained within the single dosage form. In some embodiments, the kit comprises a single dosage form, wherein a unit dose of the statin, a unit dose of vitamin D, and a unit dose of calcium are contained within the single dosage form. In some embodiments, the kit comprises multiple dosage forms. In some embodiments, the kit comprises a first dosage form comprising a unit dose of the statin, and a second dosage form comprising a unit dose of vitamin D. In some embodiments, the kit comprises a first dosage form comprising a unit dose of the statin, and a second dosage form comprising a unit dose of vitamin D and a unit dose of calcium. In some embodiments, the kit comprises a first dosage form comprising a unit dose of the statin and a unit dose of vitamin D, and a second dosage form comprising a unit dose of vitamin D. In some embodiments, the kit comprises a first dosage form comprising a unit dose of the statin, a unit dose of vitamin D, and a unit dose of calcium, and a second dosage form comprising a unit dose of vitamin D and a unit dose of calcium. In some embodiments, each dosage form is an oral dosage form. In some embodiments, the kit comprises at least 10 unit doses of the statin and at least 10 unit doses of vitamin D. In some embodiments, the kit comprises at least 30 unit doses of the statin and at least 30 unit doses of vitamin D. In some embodiments, the total amount of vitamin D to be administered daily by the one or more unit doses of vitamin D is equipotent to about 600 to about 2500 IU of ergocalciferol or cholechalciferol. In some embodiments, the total amount of vitamin D to be administered daily by the one or more unit doses of vitamin D is equipotent to about 1200 to about 2500 IU of ergocalciferol or cholechalciferol. In some embodiments, the total amount of vitamin D to be administered daily by the one or more unit doses of vitamin D is equipotent to about 600 to about 2000 IU of ergocalciferol or cholechalciferol. In some embodiments, the total amount of vitamin D to be administered daily by the one or more unit doses of vitamin D is equipotent to about 600 to about 1800 IU of ergocalciferol or cholechalciferol. In some embodiments, the total amount of vitamin D to be administered daily by the one or more unit doses of vitamin D is equipotent to about 600 to about 1500 IU of ergocalciferol or cholechalciferol. In some embodiments, the total amount of vitamin D to be administered daily by the one or more unit doses of vitamin D is equipotent to about 600 to about 1200 IU of ergocalciferol or cholechalciferol. In some embodiments, the total amount of vitamin D to be administered daily by the one or more unit doses of vitamin D is equipotent to about 600 to about 1000 IU of ergocalciferol or cholechalciferol. In some embodiments, the total amount of vitamin D to be administered daily by the one or more unit doses of vitamin D is equipotent to about 600 to about 800 IU of ergocalciferol or cholechalciferol. In some embodiments, the total amount of vitamin D to be administered daily by the one or more unit doses of vitamin D is equipotent to about 800 to about 1000 IU of ergocalciferol or cholechalciferol. In some embodiments, the total amount of calcium to be administered daily by the one or more unit doses of calcium is at least about 300 mg. In some embodiments, the total amount of calcium to be administered daily by the one or more unit doses of calcium is at least about 500 mg. In some embodiments, the total amount of calcium to be administered daily by the one or more unit doses of calcium is about 500 mg to about 2000 mg. In some embodiments, the vitamin D comprises one or more of: cholecalciferol, ergocalciferol, alfacalcidol, calcitriol, 22-oxacalcitriol, paricalcitol, doxercalciferol, and dihydrotachysterol₂. In some embodiments, the vitamin D comprises ergocalciferol. In some embodiments, the vitamin D comprises cholecalciferol. In some embodiments, the vitamin D comprises ergocalciferol and cholecalciferol. In some embodiments, the statin is metabolized by cytochrome p450(CYP) enzyme (CYP3A). In some embodiments, the statin is selected from the group consisting of: lovastatin, simvastatin, and atorvastatin. In some embodiments, the statin is atorvastatin.

In a further aspect of the invention is provided the use of the pharmaceutical compositions as described herein in the manufacture of a medicament, and particularly, in the manufacture of a medicament for use in the treatment for lowering LDL- or total cholesterol as described herein. Further, the pharmaceutical compositions thereof, as described herein, are also intended for use in the manufacture of a medicament for use in the treatment for lowering LDL- or total cholesterol and, in accordance with the methods, as described herein, unless clearly dictated otherwise by context or specifically noted.

Unless otherwise noted, the pharmaceutical compositions as described herein are intended for use in the methods of treatment as described herein and may be incorporated in the kits described herein.

### BRIEF DESCRIPTION OF THE DRAWINGS

FIG. 1A shows concentrations of atorvastatin and the two active metabolites (2-OH- and 4-OH-atorvastatin acid) vs. time after oral dosing from the non-supplemented period and during 800 IU vitamin D/day supplementation.

FIG. 1B shows concentrations of atorvastatin lactone and the inactive metabolites (2-OH- and 4-OH atorvastatin lactone) vs. time after oral dosing from the non-supplemented period and during 800 IU vitamin D/day supplementation.

FIG. 2A shows individual and mean±SE (standard error of the mean) total cholesterol concentrations from the non-supplemented (Off-D) period and during supplementation (On-D) with 800 IU vitamin D/day.

FIG. 2B shows individual and mean±SE LDL-cholesterol concentrations from the non-supplemented (Off-D) period and during supplementation (On-D) with 800 IU vitamin D/day.

FIG. 2C shows individual and mean±SE HDL-cholesterol concentrations from the non-supplemented (Off-D) period and during supplementation (On-D) with 800 IU vitamin D/day.

### DETAILED DESCRIPTION OF THE INVENTION

The invention provides methods for reducing total and/or LDL cholesterol in a human, generally comprising co-administration of a statin in combination with vitamin D. The invention also provides kits for reducing total and/or LDL cholesterol in a human, generally comprising daily dosage form(s) of a statin and vitamin D.

In vitro data have shown that active 1,25-(OH)₂ vitamin D contributes to the regulation of CYP3A activity by forming a complex with the vitamin D receptor that leads to increased transcription of CYP3A protein and greater metabolism of the CYP3A substrate midazolam. (Thummel K, et al. Mol Pharmacol. 2001;6(6):1399-406. Thompson P, et al. Biochem Biophys Res Comm. 2002;299:730-8.). The lipid lowering effects of atorvastatin are related to concentrations of atorvastatin and its hydroxy-metabolites. Because atorvastatin and its active hydroxy-metabolites are metabolized by CYP3A, pharmacologic interactions that alter CYP3A could affect therapeutic efficacy. Increased CYP3A transcription would result in greater CYP3A protein that would be predicted to increase metabolism or decrease bioavailability of CYP3A-metabolized statins, such as atorvastatin, resulting in lower statin plasma concentrations and less lipid lowering effect. Thus, co-administration of vitamin D with a CYP3A-metabolized statin would be predicted to decrease statin plasma levels, and thus statin efficacy in reducing cholesterol.

A study was designed to test the hypotheses that supplemental vitamin D intake would increase CYP3A substrate clearance in stable patients, and lower plasma concentrations of atorvastatin and its active hydroxy-metabolites, with the clinical consequence of higher total and LDL-cholesterol concentrations (Example 1). Surprisingly, it has been found that while co-administration of vitamin D with atorvastatin resulted in the predicted decrease in atorvastatin concentration, it also resulted in lower, not higher, LDL-cholesterol (and total cholesterol) concentrations. The treatment resulted in additive effects on cholesterol reduction. These findings were independent of the order of vitamin administration or subject living site, and were seen in men and women. The mean difference in LDL-cholesterol after six weeks of vitamin D supplementation was 14.4%, similar to the maximum potentially achievable with therapeutic lifestyle changes, and greater than seen with a doubling of statin doses. Without wishing to be bound by theory, the findings suggest a vitamin D effect on cholesterol metabolism or transport.

### Definitions

Unless defined otherwise or clearly indicated by context, all technical and scientific terms used herein have the same meaning as commonly understood by one of ordinary skill in the art to which this invention belongs.

Unless otherwise indicated, all numbers used in the specification and claims are to be understood as being modified in all instances by the term “about.” Accordingly, unless indicated to the contrary, the numerical parameters set forth in the following specification and attached claims are approximations that may vary depending at least upon the specific analytical technique.

“1,25-(OH)₂ vitamin D”, “1,25-dihydroxy vitamin D” are used interchangeably herein.

“25-OH vitamin D” and “25-hydroxy vitamin D” are used interchangeably herein.

“Vitamin D₂” and “ergocalciferol” are used interchangeably herein.

“Vitamin D₃” and “cholecalciferol” are used interchangeably herein.

As used herein, a “human unable to tolerate a statin when administered in a therapeutically effective dose and in the absence of vitamin D” indicates a human that suffers from sufficient negative side effects when administered the statin in a dose required for therapeutic efficacy (e.g. a dose sufficient to result in a lowering of cholesterol) that the human cannot or will not continue to take the statin due to the side effects. The side effects can be objective (e.g. elevated liver enzymes) or subjective (e.g. myalgia). Side effects of statins include, but are not limited to, elevated liver enzymes (indicating hepatic toxicity), elevated muscle enzymes, muscle damage, rhabdomyolysis, myalgia, muscle weakness, peripheral neuropathy, drug interactions with other medications undergoing metabolism by the same enzyme (in particular CYP3A (e.g. diltiazem and other nondihydropyridine calcium-channel blockers, ritonavir and other protease inhibitors, cyclosporine, erythromycin and other macrolides, azole antifungals, etc)), renal failure, actual or potential negative effects on cognitive function, actual or potential mitochondrial damage at the cellular level, and increased risk of hemorrhagic stroke.

A “therapeutically effective amount of a statin and vitamin D” as used herein indicates an amount of statin and an amount of vitamin D which in combination results in a lowering of cholesterol levels.

### Vitamin D

The vitamin D used in the invention may be any orally available form or analog of vitamin D having cholesterol-lowering effects when administered in combination with a statin, including, but not limited to, vitamin D₁ (1:1 molecular compound of ergocalciferol with lumisterol), vitamin D₂ (ergocalciferol or calciferol), vitamin D₃ (cholecalciferol), vitamin D₄ (22-dihydroergocalciferol), vitamin D₅ (sitocalciferol), calcitriol (1,25-dihydroxycholecalciferol or 1,25-(OH)₂ vitamin D₃), EB 1089 (a calcitriol analogue from Leo Pharmaceuticals), calcidiol (25-hydroxycholecalciferol or 25-OH D₃), alfacalcidol (1α-hydroxyvitamin D₃), 1α-(OH)-3-epi-vitamin D₃, maxicalcitol (22-oxa-1,25-dihydroxyvitamin D₃), oxycalcitriol (22-oxacalcitriol or OCT), falecalcitriol (1,25-(OH)₂-26,27-F₆-vitamin D₃), (20S)-1α-hydroxy-2-methylene-19-nor-bishomopregnacalciferol (2MbisP), 1α,25-dihydroxy-2-(3′-(methoxymethoxy)propylidene)-19-norvitamin D₃, 1α,25-dihydroxy-2-methylene-19-norvitamin D₃, 1,25-(OH)₂-16-ene,23-yne-vitamin D₃, 1,25-dihydroxy vitamin D₂, 25-OH vitamin D₂, doxercalciferol (1α-hydroxyvitamin D₂), dihydrotachysterol₂ (DHT₂), calcipotriol, paricalcitol (19-nor-1α,25-(OH)₂ vitamin D₂), 19-norvitamin D₂, 1,25-dihydroxy-dihydrotachysterol, 20-epi analogs from Leo Pharmaceuticals (e.g. CB1093, EB 1213 and GS1725), calcipotriene, and calcitroic acid, or a combination of any two or more (e.g. three, four, or more) of the above in any ratio. Other examples of vitamin D forms and analogs are found in New England Journal of Medicine, Jul. 24 2008, vol. 359 pages 391-403, which is herein incorporated by reference in its entirety.

The various forms and analogs of vitamin D may have differing pharmacological potencies at a given dose or amount. An amount of a vitamin D form or analog that is “equipotent” to a particular dose of ergocalciferol or cholechalciferol indicates that the amount of the form or analog has the same pharmacological potency as that particular dose of ergocalciferol or cholecalciferol. Pharmacological potency may be measured, for example, by in vitro measures (e.g. receptor binding activity or transcriptional activity (e.g. effects on mRNA expression)), by in vivo measures (e.g. parathyroid hormone suppression), or by clinical measures (e.g. change in serum 25-OH vitamin D levels, or change in cholesterol levels). For example, a particular dose of an analog of vitamin D would be equipotent to a particular dose of ergocalciferol (or cholecalciferol) if it resulted in e.g., a similar reduction in cholesterol levels. In some embodiments, equipotency is measured by transcriptional activity. In some embodiments, equipotency is measured by vitamin D receptor binding activity. In some embodiments, equipotency is measured by parathyroid hormone suppression. In some embodiments, equipotency is measured by change in serum 25-OH vitamin D levels. In some embodiments, equipotency is measured by change in cholesterol levels. Assays for measuring vitamin D receptor binding activity, transcriptional activity, parathyroid hormone suppression, serum 25-OH vitamin D levels, and cholesterol levels are known in the art. In some embodiments, equipotency is measured by comparing with ergocalciferol. In some embodiments, equipotency is measured by comparing with cholecalciferol.

The methods of the invention may comprise daily administration of one or more (e.g. two, three, four, or more) forms or analogs of vitamin D. For example, “a total daily amount of vitamin D that is equipotent to about 800 IU of ergocalciferol or cholecalciferol” can be comprised of any combination of vitamin D forms or analogs in any ratio that has the equipotent equivalent of about 800 IU total of ergocalciferol or cholecalciferol. In some embodiments, the vitamin D is selected from the group consisting of cholecalciferol, ergocalciferol, alfacalcidol, calcitriol, 22-oxacalcitriol, paricalcitol, doxercalciferol, dihydrotachysterol₂, and a combination of any two or more (e.g. three, four, or more) of the above in any ratio. In some embodiments, the vitamin D is cholecalciferol. In some embodiments, the vitamin D is ergocalciferol. In some embodiments, the vitamin D is a combination of cholecalciferol and ergocalciferol.

In general, the amount of vitamin D administered may be up to the equipotent equivalent of about 10,000 IU/day of ergocalciferol or cholecalciferol. In some embodiments, the amount of vitamin D administered may be up to the equipotent equivalent of about 5,000 IU/day of ergocalciferol or cholecalciferol. In some embodiments, the amount of vitamin D administered may be up to the equipotent equivalent of about 4,000 IU/day of ergocalciferol or cholecalciferol. In some embodiments, the amount of vitamin D administered may be up to the equipotent equivalent of about 3,000 IU/day of ergocalciferol or cholecalciferol. In various embodiments, the total daily amount of vitamin D administered by the one or more pharmaceutical compositions is equipotent to about 600 to about 2500 IU, about 700 to about 2500 IU, about 800 to about 2500 IU, about 900 to about 2500 IU, about 1000 to about 2500 IU, about 1100 to about 2500 IU, about 1200 to about 2500 IU, about 1300 to about 2500 IU, about 1400 to about 2500 IU, about 1500 to about 2500 IU, about 1600 to about 2500 IU, about 1700 to about 2500 IU, about 1800 to about 2500 IU, about 1900 to about 2500 IU, about 2000 to about 2500 IU, about 2100 to about 2500 IU, about 2200 to about 2500 IU, about 2300 to about 2500 IU, about 2400 to about 2500 IU, about 600 to about 2400 IU, about 700 to about 2400 IU, about 800 to about 2400 IU, about 900 to about 2400 IU, about 1000 to about 2400 IU, about 1100 to about 2400 IU, about 1200 to about 2400 IU, about 1300 to about 2400 IU, about 1400 to about 2400 IU, about 1500 to about 2400 IU, about 1600 to about 2400 IU, about 1700 to about 2400 IU, about 1800 to about 2400 IU, about 1900 to about 2400 IU, about 2000 to about 2400 IU, about 2100 to about 2400 IU, about 2200 to about 2400 IU, about 2300 to about 2400 IU, about 600 to about 2300 IU, about 700 to about 2300 IU, about 800 to about 2300 IU, about 900 to about 2300 IU, about 1000 to about 2300 IU, about 1100 to about 2300 IU, about 1200 to about 2300 IU, about 1300 to about 2300 IU, about 1400 to about 2300 IU, about 1500 to about 2300 IU, about 1600 to about 2300 IU, about 1700 to about 2300 IU, about 1800 to about 2300 IU, about 1900 to about 2300 IU, about 2000 to about 2300 IU, about 2100 to about 2300 IU, about 2200 to about 2300 IU, about 600 to about 2200 IU, about 700 to about 2200 IU, about 800 to about 2200 IU, about 900 to about 2200 IU, about 1000 to about 2200 IU, about 1100 to about 2200 IU, about 1200 to about 2200 IU, about 1300 to about 2200 IU, about 1400 to about 2200 IU, about 1500 to about 2200 IU, about 1600 to about 2200 IU, about 1700 to about 2200 IU, about 1800 to about 2200 IU, about 1900 to about 2200 IU, about 2000 to about 2200 IU, about 2100 to about 2200 IU, about 600 to about 2100 IU, about 700 to about 2100 IU, about 800 to about 2100 IU, about 900 to about 2100 IU, about 1000 to about 2100 IU, about 1100 to about 2100 IU, about 1200 to about 2100 IU, about 1300 to about 2100 IU, about 1400 to about 2100 IU, about 1500 to about 2100 IU, about 1600 to about 2100 IU, about 1700 to about 2100 IU, about 1800 to about 2100 IU, about 1900 to about 2100 IU, about 2000 to about 2100 IU, about 600 to about 2000 IU, about 700 to about 2000 IU, about 800 to about 2000 IU, about 900 to about 2000 IU, about 1000 to about 2000 IU, about 1100 to about 2000 IU, about 1200 to about 2000 IU, about 1300 to about 2000 IU, about 1400 to about 2000 IU, about 1500 to about 2000 IU, about 1600 to about 2000 IU, about 1700 to about 2000 IU, about 1800 to about 2000 IU, about 1900 to about 2000 IU, about 600 to about 1900 IU, about 700 to about 1900 IU, about 800 to about 1900 IU, about 900 to about 1900 IU, about 1000 to about 1900 IU, about 1100 to about 1900 IU, about 1200 to about 1900 IU, about 1300 to about 1900 IU, about 1400 to about 1900 IU, about 1500 to about 1900 IU, about 1600 to about 1900 IU, about 1700 to about 1900 IU, about 1800 to about 1900 IU, about 600 to about 1800 IU, about 700 to about 1800 IU, about 800 to about 1800 IU, about 900 to about 1800 IU, about 1000 to about 1800 IU, about 1100 to about 1800 IU, about 1200 to about 1800 IU, about 1300 to about 1800 IU, about 1400 to about 1800 IU, about 1500 to about 1800 IU, about 1600 to about 1800 IU, about 1700 to about 1800 IU, about 600 to about 1700 IU, about 700 to about 1700 IU, about 800 to about 1700 IU, about 900 to about 1700 IU, about 1000 to about 1700 IU, about 1100 to about 1700 IU, about 1200 to about 1700 IU, about 1300 to about 1700 IU, about 1400 to about 1700 IU, about 1500 to about 1700 IU, about 1600 to about 1700 IU, about 600 to about 1600 IU, about 700 to about 1600 IU, about 800 to about 1600 IU, about 900 to about 1600 IU, about 1000 to about 1600 IU, about 1100 to about 1600 IU, about 1200 to about 1600 IU, about 1300 to about 1600 IU, about 1400 to about 1600 IU, about 1500 to about 1600 IU, about 600 to about 1500 IU, about 700 to about 1500 IU, about 800 to about 1500 IU, about 900 to about 1500 IU, about 1000 to about 1500 IU, about 1100 to about 1500 IU, about 1200 to about 1500 IU, about 1300 to about 1500 IU, about 1400 to about 1500 IU, about 600 to about 1400 IU, about 700 to about 1400 IU, about 800 to about 1400 IU, about 900 to about 1400 IU, about 1000 to about 1400 IU, about 1100 to about 1400 IU, about 1200 to about 1400 IU, about 1300 to about 1400 IU, about 600 to about 1300 IU, about 700 to about 1300 IU, about 800 to about 1300 IU, about 900 to about 1300 IU, about 1000 to about 1300 IU, about 1100 to about 1300 IU, about 1200 to about 1300 IU, about 600 to about 1200 IU, about 700 to about 1200 IU, about 800 to about 1200 IU, about 900 to about 1200 IU, about 1000 to about 1200 IU, about 1100 to about 1200 IU, about 600 to about 1100 IU, about 700 to about 1100 IU, about 800 to about 1100 IU, about 900 to about 1100 IU, about 1000 to about 1100 IU, about 600 to about 1000 IU, about 700 to about 1000 IU, about 800 to about 1000 IU, about 900 to about 1000 IU, about 600 to about 900 IU, about 700 to about 900 IU, about 800 to about 900 IU, about 600 to about 800 IU, about 700 to about 800 IU, about 600 IU, about 700 IU, about 800 IU, about 900 IU, about 1000 IU, about 1100 IU, about 1200 IU, about 1300 IU, about 1400 IU, about 1500 IU, about 1600 IU, about 1700 IU, about 1800 IU, about 1900 IU, about 2000 IU, about 2100 IU, about 2200 IU, about 2300 IU, about 2400 IU, about 2500 IU of ergocalciferol or cholechalciferol.

### Statins

Suitable statins (HMGCoA reductase inhibitors) include, but are not limited to, lovastatin, pravastatin, simvastatin, atorvastatin, rosuvastatin, fluvastatin, and cerivastatin. In some embodiments, the statin is selected from the group consisting of: lovastatin, pravastatin, simvastatin, atorvastatin, rosuvastatin, and fluvastatin. In some embodiments, the statin is not mevastatin. In some embodiments, the statin is not cerivastatin. In some embodiments, the statin is not simvastatin. In some embodiments, the statin comprises a statin metabolized by cytochrome p450(CYP) enzyme (CYP3A) (e.g. lovastatin, simvastatin, atorvastatin). In some embodiments, the statin is selected from the group consisting of: lovastatin, simvastatin, and atorvastatin. In some embodiments, the statin is lovastatin. In some embodiments, the statin is simvastatin. In some embodiments, the statin is atorvastatin.

The amount of statin required to achieve a particular decrease in LDL-cholesterol or total cholesterol may be less than that required when the statin is administered in the absence of the one or more pharmaceutical compositions comprising vitamin D. Co-administration of vitamin D with the statin may result in a further reduction of total and/or LDL-cholesterol, in comparison with administration of the same dose of the statin alone. Additionally, patients who are not able to achieve a desired response in cholesterol reduction while on statins alone may be able to achieve a desired cholesterol reduction response by co-administration of statin and vitamin D. When co-administered with vitamin D, the statin dose may optionally be reduced from the dose otherwise preferred or required when administered in the absence of vitamin D, for example thus achieving similar cholesterol lowering effects but with fewer side effects or risks of side effects due to the lower statin dose. Accordingly, patients who suffer negative side effects or are at risk of negative side effects from statin administration may be able to achieve a similar cholesterol lowering effect with fewer side effects or fewer risks of side effects by combining a lower dose of statin with vitamin D. Additionally, patients who are unable to tolerate a statin due to negative side effects may be able to tolerate a lower dose of the statin, such as a dose which would not be therapeutically effective in lowering cholesterol in the absence of vitamin D; combining this lower dose of statin with vitamin D may result in a therapeutically effective amount of statin and vitamin D.

The appropriate statin total daily dose may be determined by the particular statin used, the patient's cholesterol levels and physical condition, whether the patient suffers side effects from the statin, the vitamin D total daily dose, and the like. In various embodiments, the daily dose of the statin may be: lovastatin: about 10 to about 80 mg/day, about 5 to about 40 mg/day; pravastatin: about 10 to about 40 mg/day, about 5 to about 20 mg/day; simvastatin: about 10 to about 80 mg/day, about 5 to about 40 mg/day; atorvastatin: about 10 to about 80 mg/day, about 5 to about 40 mg/day; rosuvastatin: about 5 to about 40 mg/day, about 2.5 to about 20 mg/day; fluvastatin: about 20 to about 80 mg/day, about 10 to about 80 mg/day, about 10 to about 40 mg/day.

### Additional Therapeutic Agents

One or more (e.g. two, three, four, five, or more) additional therapeutic agent(s) may optionally be administered in conjunction with the vitamin D and statin treatment regimens described herein. Suitable therapeutic agents include, but are not limited to: calcium, multivitamin formulations, non-statin lipid lowering agents, non-statin HDL-raising agents, insulin, and non-insulin diabetic agents. Non-statin lipid lowering agents include, for example, bile acid sequestrants (e.g. cholestyramine, colestipol, colesevelam), fibric acid derivatives (e.g. gemfibrozil, fenofibrate), omega-3 fatty acids (e.g. OMACOR®, fish oil capsules), niacin, and cholesterol absorption inhibitors (e.g. ezetimide). Non-statin HDL-raising agents include, for example, cholesteryl ester transfer protein (CETP) inhibitors. Non-insulin diabetic agents include, for example, sulfonylureas (e.g. tolbutamide, acetohexamide, tolazamide, chlorpropamide, glipizide, glyburide, glimepiride, gliclazide), biguanides (e.g. metformin, phenformin), meglitinides (e.g. repaglinide, nateglinide), thiazolidinediones (e.g. glitazones such as rosiglitazone, pioglitazone, etc.), 6-alpha-glucosidase inhibitors (e.g. miglitol, acarbose), glucagon-like peptide (GLP) analogs (e.g. exenatide, liraglutide), and gastric inhibitory peptide (GIP) analogs (e.g. DPP-4 inhibitors such as vildagliptin and sitagliptin). In some embodiments, the one or more additional therapeutic agents includes a non-statin lipid lowering agent. In some embodiments, the one or more additional therapeutic agents includes a HDL-raising agent. In some embodiments, the one or more additional therapeutic agents includes insulin. In some embodiments, the one or more additional therapeutic agents includes a non-insulin diabetic agent. In some embodiments, the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog. In some embodiments, one or more additional therapeutic agents include ezetimide. In some embodiments, a microsomal triglyceride transfer protein (MTP) inhibitor is not administered in conjunction with the vitamin D and statin treatment.

Calcium (e.g. oral dosage forms of calcium such as calcium carbonate, calcium phosphate, and calcium citrate) may generally be administered as a total daily amount of at least about 300 mg. In some embodiments, the total daily amount of calcium administered is at least about 500 mg. In some embodiments, the total daily amount of calcium administered is at least about 800 mg. In various embodiments, the total daily amount of calcium administered is about 300 to about 2000 mg, about 500 to about 2000 mg, about 700 to about 2000 mg, about 800 to about 2000 mg, about 1000 to about 2000 mg, about 1200 to about 2000 mg, about 1500 to about 2000 mg, about 500 to about 1800 mg, about 500 to about 1500 mg, about 500 to about 1200 mg, about 700 to about 1500 mg, about 700 to about 1200 mg, about 800 to about 1000 mg, about 300 mg, about 400 mg, about 500 mg, about 600 mg, about 700 mg, about 800 mg, about 900 mg, about 1000 mg.

### Dosage Forms & Administration

One or more dosage forms (e.g. two, three, four, or more) in one or more (e.g. two, three, four, or more) daily doses may be used to administer the vitamin D, statin, and optional one or more additional therapeutic agents. In some embodiments, the vitamin D is administered in a single daily dose. In some embodiments, the vitamin D is administered in two daily doses. In some embodiments, the vitamin D is administered in more than two daily doses. In some embodiments, the statin is administered in a single daily dose. In some embodiments, the statin is administered in two daily doses. In some embodiments, the statin is administered in more than two daily doses. The additional therapeutic agent(s) may be administered in less than once daily doses (e.g. every other day, once per week, etc.), a once daily dose, twice daily doses, or more than twice daily doses, as appropriate for the particular therapeutic agent.

One or more dosage forms (e.g. two, three, four, or more) may be used in the methods of the invention. For example, each of the vitamin D, statin, and one or more optionally therapeutic agents may be administered in separate dosage forms and/or in one or more combined dosage forms. For example, the vitamin D may be combined with the statin in a single dosage form, may be administered in separate dosage forms, or may be administered in both separate and combined dosage forms (e.g. a vitamin D-only dosage form once per day, and a vitamin D and statin combined dosage form once per day). The one or more additional therapeutic agent(s) may each be administered, for example, in one or more combined dosage forms (e.g. combined with another agent, with vitamin D, and/or with a statin) and/or in one or more separate dosage forms. For example, the therapeutic agent(s) may each be combined in a dosage form with vitamin D, may be combined in a dosage form with a statin, may be combined in a dosage form with vitamin D and a statin, and/or administered in a separate dosage form.

The dosage forms comprising vitamin D, statin, and/or optional one or more therapeutic agents may be formulated into pharmaceutical compositions. The pharmaceutical compositions comprising vitamin D and statin are formulated for oral administration, such as tablets, pills, powders, lozenges, sachets, cachets, elixirs, suspensions, emulsions, solutions, syrups, and the like. The optional one or more therapeutic agents may be formulated in pharmaceutical compositions suitable for administration by a variety of routes including oral, parenteral, transdermal, topical, rectal, and intranasal, when suitable for the particular therapeutic agent. The pharmaceutical compositions may be formulated with one or more pharmaceutically acceptable carriers, and may comprise one or more excipients, binding agents, lubricating agents, wetting agents, emulsifying and suspending agents, preserving agents, sweetening agents, flavoring agents, and the like. The doses of the vitamin D, statin, and optional one or more therapeutic agents may be determined by a physician, in the light of the relevant circumstances, including the condition to be treated, the severity of the condition being treated, the chosen route of administration, the actual compound administered, the age, weight, and response of the individual patient, the severity of the patient's symptoms, and the like. The treatment regimens of the invention may further be combined with therapeutic lifestyle modifications such as those recommended by the American Heart Association, including dietary counseling and restriction, exercise, and weight loss in the presence of obesity.

In some embodiments, at least a portion of the daily dose of vitamin D is administered as a multivitamin. In some embodiments, the entire daily dose of vitamin D is administered as a multivitamin. In some embodiments, at least a portion of the daily dose of vitamin D is administered in a combined dosage form with calcium. In some embodiments, the entire daily dose of vitamin D is administered in a combined dosage form with calcium. For example, in some embodiments, about 300 to about 900 IU of vitamin D are administered once per day in a separate dosage form, and an additional about 300 to about 900 IU of vitamin D are administered once per day in a multivitamin. In some embodiments, about 300 to about 900 IU of vitamin D in a combined dosage form with calcium are administered once per day, and an additional about 300 to about 900 IU of vitamin D are administered once per day in a multivitamin. In some embodiments, about 400 IU of vitamin D are administered once per day in a multivitamin, and a dosage form comprising 200 IU vitamin D plus 500 mg of calcium are administered twice per day.

Any of the pharmaceutical compositions comprising any of the dosage forms described herein may be packaged into kits, and may include instructions for administration of the pharmaceutical compositions according to the methods of the invention as described herein.

The treatment regimen may be administered for at least about 6 weeks. In various embodiments, the treatment regimen is administered for at least about 8 weeks, at least about 10 weeks, at least about 12 weeks, at least about 15 weeks, at least about 20 weeks, at least about 4 months, at least about 6 months, at least about 1 year.

The vitamin D and statin treatment regimen results in a lowering of total cholesterol and/or LDL cholesterol. The lowering of total and/or LDL cholesterol may occur without other changes in the human's diet, lifestyle, or concomitant medications. In various embodiments, the treatment regimen results in a reduction of total cholesterol of at least about 5%, at least about 6%, at least about 7%, at least about 8%, at least about 9%, at least about 10%, at least about 11%, at least about 12%, at least about 13%, at least about 14%, at least about 15%, at least about 18%, at least about 20%, at least about 25%, after about six weeks, about eight weeks, about twelve weeks, about 4 months, about 6 months, about 12 months of treatment. In various embodiments, the treatment regimen results in a reduction of LDL-cholesterol of at least about 5%, at least about 6%, at least about 7%, at least about 8%, at least about 9%, at least about 10%, at least about 11%, at least about 12%, at least about 13%, at least about 14%, at least about 15%, at least about 18%, at least about 20%, at least about 25%, after about six weeks, about eight weeks, about twelve weeks, about 4 months, about 6 months, about 12 months of treatment.

In some embodiments, the treatment regimen results in increased circulating 25-OH vitamin D (combined 25-OH vitamin D₂ and 25-OH vitamin D₃). Circulating vitamin D levels may be measured in, for example, serum or plasma. In some embodiments, the circulating vitamin D levels are measured in serum. In some embodiments, the circulating vitamin D levels are measured in plasma. In general, when measured using mass spectrometry, total 25-OH vitamin D (combined 25-OH vitamin D₂ and 25-OH vitamin D₃) serum concentrations below 20 ng/mL are considered vitamin D deficiency, levels of 20-30 ng/mL are interpreted as vitamin D insufficiency. In some embodiments, the serum levels of 25-OH vitamin D (combined 25-OH vitamin D₂ and 25-OH vitamin D₃) are measured according to liquid chromatography tandem mass spectrometry (LCMSMS) by the Quest Diagnostics protocol.

The methods of the invention may be used to treat any human in need thereof. In some embodiments, the human is female. In some embodiments, the female is premenopausal. In some embodiments, the human is male. In some embodiments, the human has congestive heart failure. In some embodiments, the human does not have congestive heart failure. In some embodiments, the human is a pre-menopausal female with congestive heart failure. In some embodiments, the human is a pre-menopausal female without congestive heart failure. In some embodiments, the human is a male with congestive heart failure. In some embodiments, the human is a male without congestive heart failure. In some embodiments, the human is unable to tolerate the statin when administered in a therapeutically effective dose and in the absence of vitamin D. In various embodiments, the human is at least about 12 years of age, at least about 20 years of age, at least about 30 years of age. In some embodiments, the human does not have psoriasis. In some embodiments, the human does not have osteoporosis. In some embodiments, the human is not at risk for bone fractures. In some embodiments, the human does not have multiple sclerosis. In some embodiments, the human is diabetic. In some embodiments, the human is not diabetic. In some embodiments, the human does not have an autoimmune disorder. In some embodiments, the human does not have rheumatoid arthritis. In some embodiments, the human does not have lupus. In some embodiments, the human does not have an inflammatory bowel disease. In some embodiments, the human does not have Crohn's disease. In some embodiments, the human does not have ulcerative colitis. In some embodiments, the human does not have a renal disorder. In some embodiments, the human does not have an immunoproliferative skin disorder. In some embodiments, the human does not have cancer. In various embodiments, the human has not previously been treated with at least about 600 IU per day of ergocalciferol or cholecalciferol or equipotent amount of other vitamin D form or analog thereof for at least about 6 weeks, at least about 8 weeks, at least about 10 weeks, at least about 12 weeks, at least about 15 weeks, at least about 20 weeks, at least about 4 months, at least about 6 months, at least about 1 year. In some embodiments, prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy. In some embodiments, prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy. In some embodiments, after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy. In some embodiments, after at least about 12 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy.

The invention is further illustrated by the following non-limiting examples. Variations and modifications may be made in respect of the methods and kits as above described without departing from the scope of the invention as described and as defined in the claims.

### EXAMPLES

### Example 1

Summary

Vitamin D supplementation effects on concentrations of atorvastatin and cholesterol in patients were investigated. Briefly, sixteen patients (8 men, 8 women; 10 Caucasian, 4 African American, 1 Hispanic, 1 Asian), aged 63±11 yr (mean±S.D., weight 92±31 kg) on atorvastatin (45±33 mg/day) were studied with and without supplemental vitamin D (800 IU/day for six weeks, with 400 IU via multivitamin (as ergocalciferol), and 400 IU (as cholecalciferol) with 1000 mg calcium). Diet was not altered and concomitant medications remained the same throughout the study. The order of study was: a) “Off-D” following by “On-D” in 10 patients, and b) “On-D” followed by “Off-D” in six patients. Levels of vitamin D (1,25-dihydroxy and 25-OH-metabolites of D₂ and D₃), atorvastatin (parent and OH-acid metabolites) at 0.5, 3 and 10 hours after dosing, and cholesterol (total, LDL-cholesterol and HDL-cholesterol) were determined. Vitamin D supplementation increased vitamin D-25-OH-metabolites (p<0.0001) without increased 1,25-dihydroxy vitamin D. Atorvastatin concentrations were lower during vitamin D supplementation (On-D) (p<0.001). LDL-cholesterol and total cholesterol were lower On-D (97±28 mg/dL vs. 83±30 and 169±35 mg/dL vs. 157±37, respectively, p<0.005). Vitamin D supplementation lowered atorvastatin and active metabolite concentrations with additive effects on cholesterol concentrations.

Methods

Subject selection. Subjects of any age, any race, and both sexes taking atorvastatin for over six weeks at a stable dosage were recruited. Exclusion criteria were clinical instability of underlying disease processes (ex. recent hospitalization, medication dose changes within two weeks, or new medications within one month), inability to give informed consent, recent transfusion, severe renal failure or dialysis, or contraindications to calcium administration (hypercalcemia, hyperparathyroidism), recent fracture, severe osteoporosis (by diagnosis or administration of bisphosphonates), feeding tube, intestinal bypass surgery, inability to swallow calcium tablets, or history of adverse reactions to calcium, vitamin D or multivitamins. Historical and medically related data were obtained from participants, a full medication inventory and over the counter medications and nutraceutical use was verified by visual inspection of medications and nutraceuticals at study entry and the end of each six week study period.

Study protocol. A prospective crossover study was performed. The overall design was to measure vitamin D, atorvastatin, and lipid concentrations in patients under two conditions: 1) After six weeks of 800 IU supplemental vitamin D/day from a multivitamin containing 400 IU (as ergocalciferol) and two calcium (500 mg) plus 200 IU vitamin D (as cholecalciferol) tablets/day (“On-D”); and 2) After six weeks of no supplemental vitamin D in any form (“Off-D”). Study order was determined by subject vitamin intake at enrollment: if subjects were not taking any form of vitamin D at enrollment they were studied in the order “Off-D” followed by “On-D” (n=10). If subjects were taking vitamin D supplementation at recruitment, assigned study order was “On-D” after six week of standardized study dispensed vitamins followed by the “Off-D” study after six weeks of vitamin D discontinuation. In this arm, three participants who had been prescribed calcium with vitamin D at study entry continued calcium (500 mg twice daily) during the “Off-D” period; three did not. Multivitamins and calcium were dispensed to all subjects from one manufacturers lot (multivitamin as Therapeutic-M Tablets from Goldline Laboratories, Inc, Miami Fla. 33137 with 400 IU vitamin D as ergocalciferol; vitamin D plus calcium as Oyster Shell Calcium with vitamin D (500 mg+200 IU vitamin D as cholecalciferol), Major Pharmaceuticals, Livonia Mich. 48150; calcium only as Calcium Carbonate Tablets (1250 mg) Boehringer Ingelheim, Roxane Laboratories, Inc. Columbus Ohio 43216). All other prescribed medications were continued unchanged throughout the study. Dosing of atorvastatin (with dose held constant throughout the study) was at night/bedtime in 15 of 16 subjects and in the morning in one subject.

Participants were contacted every two weeks to assess potential adverse effects and adherence. At the end of each six week period, vitamin D (total and hydroxy-metabolites), atorvastatin (and hydroxy-metabolites), and cholesterol (total, LDL-cholesterol, and HDL-cholesterol) concentrations were measured, medication diaries were completed for the three days prior to blood sampling in outpatients and medication dispensing records reviewed for nursing home residents, and pill counts for vitamin D and multivitamins were performed. Blood (10 cc) was drawn for atorvastatin concentration determinations at 0.5, 3, and 10 hours after atorvastatin dosing; 10 cc blood was drawn for lipid determinations (after fasting in 12 of 16 subjects), and 5 cc blood was drawn for determination of vitamin D and 25-OH metabolite concentrations. Thirteen subjects were admitted to the UCSF Clinical Translational Research Institute (CTSI) Clinical Research Center for overnight sampling in 12 and daytime sampling in one; three were studied at the Jewish Home, San Francisco during overnight studies. Samples and data sent to laboratories for analysis were blinded as to study phase. Adherence was estimated as number doses consumed divided by the number of doses prescribed.

Vitamin D concentration data. Serum samples were analyzed for total 1,25-dihydroxy vitamin D (the active forms of vitamin D from ergocalciferol and cholecalciferol (1,25-dihydroxy vitamin D₂ and 1,25-dihydroxy vitamin D₃, respectively)), total and individual 25-hydroxy vitamin D forms (25-hydroxy vitamin D₂ and 25-hydroxy vitamin D₃) by Nichols Institute, San Juan Capistrano, Calif. using liquid chromatography tandem mass spectrometry (LCMSMS) according to the Quest Diagnostics protocol.

Atorvastatin concentration data. Atorvastatin and its main metabolites were measured in plasma using a liquid chromatography/triple quadropole tandem mass spectrometry system, consisting of a 717 plus autosampler (Waters Corporation, Milford, Mass.), and Quattro Ultima (Micromass, Manchester, UK) detector with electrospray positive ionization mode by the Drug Studies Unit, UCSF. The multiple reaction monitor was set at Atorvastatin: 559.1-440.2 m/z, 2- or 4-Hydroxy Atorvastatin: 575.2-440.3 m/z, Atorvastatin Lactone: 541.2-448.3 m/z, 2- or 4-Hydroxy Atorvastatin Lactone: 557.2-448.3 m/z, Atorvastatin-D5 (IS for ATV, 2-OH-ATV, 4-OH-ATV): 564.2-445.3 m/z, Atorvastatin-D5 Lactone (IS for ATV-Lactone, 2-OH-ATV-Lactone, 4-OH-ATV-Lactone): 546.2-453.2 m/z. The sample cone voltage and collision energy for all analytes and internal standard were set at 30 V and 20 eV, respectively. Chromatography was performed on BDS C18 column (50×4.6 mm, 5 μm particle size, Thermo Electron Corporation, Bellefonte, Pa.). The mobile phase A was 20% acetonitrile containing 0.05% acetic acid and 5 mM ammonium acetate and mobile phase B was 80% acetonitrile containing 0.05% acetic acid and 5 mM ammonium acetate. The gradient elution time program was set as follows: 0-1.5 min, B, 20-100%; 1.5-4.0 min, B, 100%; 4.0-4.5 min, B, 100-20%. The flow rate was 1.0 ml/min from 0 to 4.5 min and increased to 1.5 ml/min at 4.6 min and kept at 1.5 ml/min from 4.6 to 6.5 min. Run time for each sample was 6.5 min. Twenty-five percent of flow liquid was split into the mass system. Retention times were as follows: Atorvastatin and D5-Atorvastatin, 3.8 min; 2-Hydroxy Atorvastatin, 3.7 min; 4-Hydroxy Atorvastatin, 3.1 min; Atorvastatin Lactone and D5-Atorvastatin Lactone, 4.2 min; 2-Hydroxy Atorvastatin Lactone, 4.1 min; 4-Hydroxy Atorvastatin Lactone, 3.6 min. Samples and standards were prepared as described in (Jemal M, et al. Quantitation of the acid and lactone forms of atorvastatin and its biotransformation products in human serum by high-performance liquid chromatography with electrospray tandem mass spectrometry. Rapid Commun. Mass Spectrom. 1999;13:1003-15.) Human plasma (0.5 ml) was acidified with adding 0.5 ml of chilled 0.1 M Sodium Acetate buffer (pH 5.0), then extracted with 3 ml of Methyl Tert-Butyl Ether. The organic layer then transferred to another tube and evaporated by N₂ gas and reconstituted with 0.2 ml 50% CH₃CN containing 0.05% acetic acid and 5 mM ammonium acetate, and 20 μl was injected onto column. Lower limit of detection was 0.313 ng/ml for atorvasatin and metabolites. Standard curves (atorvastatin calcium salt, 2-hydroxy atorvastatin dihydrate monosodium salt, 4-hydroxy atorvastatin disodium salt, TRC, North York, Ontario, Canada) were linear over the range from 0.313 to 40 ng/ml with sample dilution for initial values out of this range. Internal standard was D5-atorvastatin sodium salt. Inter-day precision and accuracy were coefficient of variation (CV) of 2-5%, 2-7%, 3-9%, 3-9%, 5-12, 6-9%, and relative error (RE) of −1 to 6%, 0.1-7%, -5 to 5, 1 to 11%, -5 to 10, 3-7% for atorvastatin, atorvastatin lactone, 2-OH atorvastatin acid, 2-OH atorvastatin lactone, 4-OH-atorvastatin acid, and 4-OH-atorvastatin lactone, respectively. Intraday precision and accuracy were CV of 3-4%, 1-2%, 2-5%, 2-3%, 3-5% and 4-5%; and RE range was −2 to −0.4, 4-6%, −7 to −3, −1 to 0%, −8 to 3%, and −6 to 1% for atorvastatin, atorvastatin lactone, 2-OH atorvastatin acid, 2-OH-atorvastatin lactone, 4-OH-atorvastatin acid, 4-OH-atorvastatin lactone, respectively. Recovery was 89% for atorvastatin, 91% for atorvastatin lactone, 89% for 2-OH atorvastatin acid, 91% for 2-OH atorvastatin lactone, 56% for 4-OH-atorvastatin acid, and 98% for 4-OH atorvastatin lactone. Area under the plasma concentration vs. time curve (AUC) was calculated by trapezoidal rule.

Lipid concentration data. Serum samples were analyzed for cholesterol, LDL-cholesterol (calculated) and HDL-cholesterol concentrations by enzymatic assay (Quest Diagnostics, San Jose, Calif.).

Dietary analyses. Dietary vitamin D and cholesterol intake was estimated by Block 2005 Food Frequency 1 questionnaire (NutritionQuest, Berkeley, CA) (Block G, et al. A data-base approach to diet questionnaire design and testing. Am J Epidemiol. 1986;124:453-69. Block G, et al. A reduced dietary questionnaire: development and validation. Epidemiology. 1990;1(1):58-64) in community-dwellers, and analysis of three-day non-consecutive food intake records in nursing home residents completed by nursing assistants and research staff. Intakes were quantified in household measurements and documented. A single, trained individual in the UCSF CTSI entered data into Food Processor SQL Nutrition Analysis Software from EHSA Research, Salem, Oreg.©, 2006-07. Mean values for nutrients were obtained.

Other Measurements. Weight and height were measured using balance beam scale. Body mass index was calculated as weight (kg) divided by height² (M). Blood pressure and heart rate were measured using automated aneroid devices.

Statistical analysis. Data are mean±S.D. unless otherwise noted. Statistical analyses were done using STATVIEW©, SAS, Institute, Inc, Berkeley, Calif. Between period comparisons of concentration data (raw and AUC) and ratios were by paired t. Vitamin D concentration data were compared by paired t, except for D₂ concentration analyses analyzed by nonparametric paired sign test to include non-detectable values without supplementation. Tests for effects of vitamin supplementation, order of administration, and sex on cholesterol concentrations were by ANOVA for repeated measures. Linear regression was used to test for relationships between variables (vitamin D concentration, atorvastatin, cholesterol, LDL-cholesterol, change in cholesterol).

Results

Study Population. Seventeen subjects consented and sixteen subjects were enrolled and completed the study. Mean (±S.D.) age was 62.8±11 years (range 49-86 yr), weight was 91.5±30.8 kg (range 57.2-168.9 kg), height was 168±10 cm (range 144-181.3 cm), BMI was 32.7±11.6 (range 23.1-64.8), lean body weight was 53.6±9.2 kg (range 38.4-69.7 kg). Eight were men (6 Caucasian (1 nursing home resident), 1 African American, 1 Asian) and eight were women (4 Caucasian (2 nursing home residents), 3 African American, 1 Hispanic). Diagnoses included hypertension in nine, diabetes in six, coronary artery disease in three, moderate renal disease in three. Average number of medications consumed per subject per day was eight (range 3-14). Concomitant medications taken by >1 subject were aspirin in nine, antidepressants in six (selective serotonin reuptake inhibitors in two, serotonin and norepinephrine reuptake inhibitors in two), angiotensin converting enzyme inhibitors in five, angiotensin receptor blockers in five, beta blockers in five, diuretics in five, oral hypoglyemic agents in five, histaminergic blockers in three, insulin in three, proton pump inhibitors in three, calcium channel antagonists in three, opiates in two, clopidogrel in two. One took fexofenadine. One took diltiazem, a recognized CYP3A4 inhibitor. (Flockhart D. http://www.drug-interactions.com. Wilkinson G. Drug metabolism and variability among patients in drug response. N Engl J Med. 2005;352(21):2211-21.) None took recognized CYP3A4 inducers. Four (three men and one woman) were smokers (average of half pack per day, range 0.25-2 packs per day), six consumed alcohol at least monthly.

Vitamin D Data. No difference was detected in active 1,25 dihydroxy vitamin D or vitamin D₃ 25-hydroxy metabolite concentrations between the “on-D” and “off-D” study periods. Total 25-hydroxy vitamin D concentrations were higher during supplementation compared to the off-D period (p<0.0001), due chiefly to higher 25-hydroxy vitamin D₂ (p<0.007), Table 1.

Vitamin D₃ and its 25-hydroxy metabolite reflect exogenous intake from foods of animal origin and endogenous ultraviolet light-stimulated conversion in the skin. Only small amounts of vitamin D₂ are obtained from food sources and the vitamin D₂, 25-hydroxy metabolite is usually an indicator of exogenous sources such as supplementation. The increases in D₂, 25-hydroxy serum concentrations seen in this study are evidence of adherence to vitamins. Levels of 25-OH vitamin D₂ of ≧4 ng/ml are considered to reflect supplementation. One subject had concentrations <4 ng/ml during supplementation, suggesting non-adherence. Four participants had 25-OH vitamin D₂ concentrations >4 ng/mL during the “off-D” period, suggesting non-study-related supplementation.

Some degree of vitamin D deficiency was found in the majority of patients in the absence of vitamin D supplementation.: Nine of fourteen participants had total 25-OH vitamin D concentrations <20 ng/mL and four had levels between 20-30 ng/mL off vitamin D supplementation (data not obtained in 2 due to sampling or processing error). Total 25-OH vitamin D concentrations below 20 ng/mL (with this assay) are considered vitamin D deficiency, levels of 20-30 ng/mL are interpreted as vitamin D insufficiency.

After six week supplementation, several continued to have biochemical evidence of vitamin D deficiency or insufficiency: During supplementation, two of 15 participants had 25-OH vitamin D levels <20 ng/mL, although the levels were higher than without vitamin D supplementation in both. Eight of 15 had levels between 20-30 ng/mL on supplemental vitamin D, increased from the non-supplemented period in seven.

The time periods of vitamin supplementation and discontinuation were based on the time required to reach steady-state concentrations and activity of CYP3A and atorvastatin (Shaefer E, et al. Effects of atorvastatin on fasting and postprandial lipoprotein subclasses in coronary heart disease patients versus control subjects. Am J Cardiol. 2002;90:689-96. Shaefer E, et al. Comparisons of effects of statins (atorvastatin, fluvastatin, lovastatin, pravastatin, and simvastatin) on fasting and postprandial lipoproteins in patients with coronary heart disease versus control subjects. Am J Cardiol. 2004;93:31-9.) but may not have reached vitamin D steady-state conditions or full repletion of deficiencies.

Adherence assessed by pill counts was 99±10% overall (all study periods, all subjects) with adherence for dispensed multivitamins of 104±10% (range 96-133), calcium plus vitamin D of 99±4% (range 91-106), and 96±3% for calcium (n=3; range 94-99).

Dietary Intake. Mean dietary intake of vitamin D was estimated at 158±123 IU/day (range 25-407 IU/day), representing intake when “off-D”. Total vitamin D intake during the “on-D” phase was 958±123 IU/day (range 825-1207 IU/day). Mean cholesterol intake was 272±245 mg/day (range 87-972 mg/day).

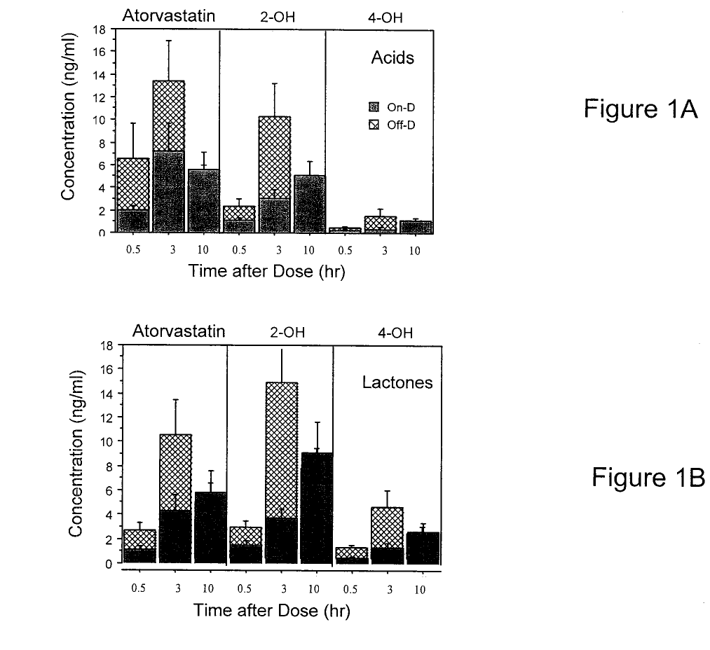

Atorvastatin. Atorvastatin was prescribed as LIPITOR®. Adherence was 100% for the three days before studies by medication diary and/or nursing records. Mean dose was 45±33 mg/day at night/bedtime in 15 of 16 participants. The subject receiving atorvastatin in the morning also received the CYP3A inhibitor diltiazem. Data from this subject are excluded from mean concentration and AUC data. AUC's for atorvastatin and its two active metabolites (2-hydroxy-atorvastatin acid and 4-hydroxy-atorvastatin acid) were significantly lower during vitamin D co-administration compared to the off-D study period, as hypothesized (see Table 1 and FIGS. 1A-1B). Concentrations of atorvastatin, and the two active metabolites, 2-OH- and 4-OH-atorvastatin acid, are shown vs. time after oral dosing from the non-supplemented period and during 800 IU vitamin D/day supplementation in the upper panel (FIG. 1A). Concentrations of atorvastatin lactone and the inactive metabolites, 2-OH- and 4-OH atorvastatin lactone are shown vs. time in the lower panel (FIG. 1B). Data from the Off- D period are represented by cross-hatched bars, data from the On-D period are represented by solid bars of light grey. Data are mean±SE (standard error of the mean).

Ratios of hydroxy-atorvastatin acids to atorvastatin did not differ between study periods (2-hydroxy-atorvastatin acid to atorvastatin ratios were 0.81±0.32 off-D compared to 0.74±0.36 during supplemental vitamin D; 4-hydroxy-atorvastatin acid ratios were 0.10±0.09 off-D compared to 0.09±0.09 during supplemental vitamin D); nor did ratios between atorvastatin acid and atorvastatin lactone differ at 0.5, 3 or 10 hours after dosing between the two study periods. While the relationship between active vitamin D (1,25-(OH)₂ vitamin D) concentrations and total active atorvastatin concentrations did not reach statistical significance, the trend was toward smaller total active atorvastatin AUC at higher active vitamin D concentrations (p=0.10).

The lowering of parent drug as well as active metabolites suggests either lower bioavailability or faster clearance/metabolism. Because both atorvastatin and its active hydroxy-acid forms are metabolized by CYP3A, the most likely explanation is increased CYP3A-mediated clearance in either the gut or liver.

Three subjects received calcium alone during the “off-D” phase and decreases in atorvastatin concentrations were seen in these subjects during the “On-D” phase compared to “Off-D”, suggesting that calcium is not the responsible agent.

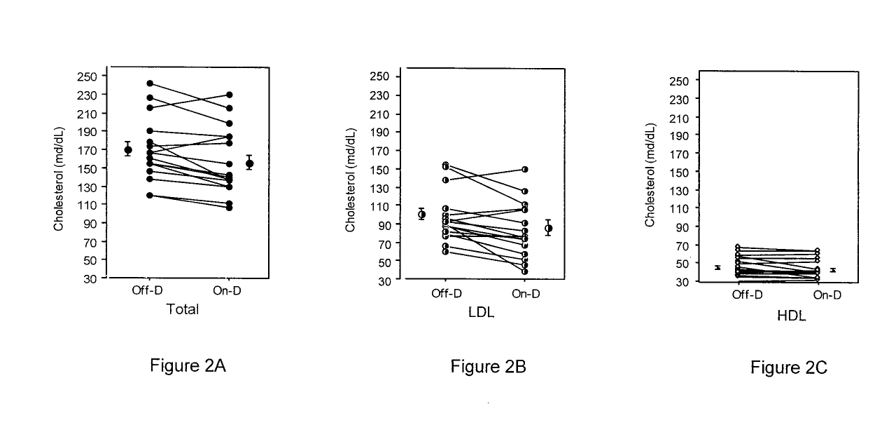

Cholesterol Data. Group data are in Table 1 and individual data are in FIGS. 2A-2C. Individual and mean±SE Total cholesterol, LDL-cholesterol, and HDL-cholesterol concentrations from the non-supplemented (Off-D) period and during supplementation (On-D) with 800 IU vitamin D/day are shown in the left (FIG. 2A), middle (FIG. 2B), and right (FIG. 2C) panels, respectively. Total cholesterol was lower during supplemental vitamin D in thirteen of sixteen subjects resulting from lower LDL-cholesterol levels during vitamin D (p<0.005). No difference in HDL-cholesterol was detected with vitamin D supplementation. No order of administration, or sex-cholesterol effect interactions were detected.

No linear relationship was detected between active vitamin D concentrations (1,25-dihydroxy vitamin D) and cholesterol or LDL-cholesterol concentrations; nor, was the change in cholesterol concentrations during vitamin D supplementation linearly related to the baseline vitamin D levels or the changes in vitamin D levels. Two of three subjects without lower cholesterol and LDL-cholesterol concentrations during the “On-D” compared to the “Off-D” period had detectable levels of vitamin D₂ 25-hydroxy concentrations during the assigned “Off-D”, suggesting vitamin D supplementation during the Off-D period. In the third, D₂ 25-hydroxy vitamin D was not measured during the “Off-D” period.

TABLE 1
Vitamin D, Atorvastatin, and Cholesterol Concentrations during and without
vitamin D supplementation.

| | | vitamin D | No vitamin D | | °Statistical |
|---|---|---|---|---|---|
| | | Supplementation | Supplementation | **N | Significance |
| Vitamin D | 1,25-dihydroxy | 38 ± 15 | 37 ± 17 | 13 | ns |
| (active) | (pg/mL) | | | | |
| Vitamin D | Total 25- | 29 ± 14 | 19 ± 18 | 14 | p < 0.0001 |
| (25-OH) | hydroxy (ng/mL) | | | | |
| | D₂, 25-hydroxy | 10 ± 3 (n = 13) | <4 (n = 10) | 14 | p < 0.002 |
| | (ng/mL) | <4 (n = 1) | 9 ± 4 (n = 4) | | |
| | D₃, 25-hydroxy | 20 ± 17 | 17 ± 19 | 14 | ns |
| | (ng/mL) | | | | |
| Atorvastatin | AUC 0.5 hr-10 hr | 3250 ± 3037 | 5190 ± 4557 | 15 | p < 0.005 |
| | (ng/mL * min) | | | | |
| 2-OH | AUC 0.5 hr-10 hr | 1941 ± 1417 | 4042 ± 3677 | 15 | p < 0.008 |
| 4-OH | (ng/mL * min) | | | | |
| Total Active | AUC 0.5 hr-10 hr | 314 ± 337 | 690 ± 884 | 15 | p < 0.05 |
| | (ng/mL * min) | | | | |
| | AUC 0.5 hr-10 hr | 5505 ± 4555 | 9923 ± 8834 | 15 | p < 0.005 |
| | (ng/mL * min) | | | | |
| Metabolite/ | AUC 2-OH + | 0.83 ± 0.43 | 0.91 ± 0.385 | 15 | ns |
| Parent Ratio | 4-OH/AUC | | | | |
| | Atorvastatin | | | | |
| Cholesterol | Total (mg/dL) | 157 ± 37 | 169 ± 35 | 16 | p < 0.005 |
| | LDL-cholesterol | 83 ± 30 | 97 ± 28 | 16 | p < 0.005 |
| | (mg/dL) | | | | |
| | HDL-cholesterol | 44 ± 11 | 47 ± 11 | 16 | ns |
| | (mg/dL) | | | | |

**N = Number of subjects for paired data comparisons.
Data are mean ± S.D.
ns represents not statistically significant.
n = number of subjects for computation;
AUC = area under the concentration vs. time curve.
2-OH = 2-hydroxyatorvastatin acid,
4-OH = 4-hydroxyatorvastatin acid.
Total Active = atorvastatin plus 2-OH and 4-OH atorvastatin acid.
°Analyses were ANOVA for repeated measures except for nonparametric rank testing for D₂,
25-OH vitamin D concentrations.

Other Measurements. Measurements during vitamin D supplementation were: systolic blood pressure of 125±12 mmHg, diastolic blood pressure 69±8 mmHg, mean arterial pressure 88±8 mmHg, and heart rate of 65±15 bpm. Measurements Off -D were: systolic blood pressure 130±14 mmHg, diastolic blood pressure 72±8 mmHg, mean arterial pressure 91±8 mmHg, and heart rate 81±14 bpm. No significant differences in blood pressure or heart rate were detected between the study phases.

Adverse Effects. Constipation was reported by two subjects during the “On-D” period with constipation during the “Off-D” period in the one assigned to calcium during this period. Constipation was chronic in one and irritable bowel syndrome was a chronic condition in the other.

### Example 2

A study similar to that described in Example 1 is performed, with atorvastatin and 1000 IU/day of either vitamin D₂ or vitamin D₃ or placebo for a period of 12 weeks.

Briefly, apparent oral clearance of atorvastatin and cholesterol concentrations in medically stable patients receiving atorvastatin with and without supplemental vitamin D (either D₂ or D₃ or identical placebo) intake is evaluated. The focus is on clinical populations including the elderly, frail, and minorities under steady-state conditions and with conditions reflecting “usual” clinical conditions with use of sparse sampling techniques and minimal impact on clinical care. Vitamin D and lipid concentrations are measured at baseline, 6 and 12 weeks, and atorvastatin CL/F (clearance/bioavailability) at baseline and 12 weeks. About 90 participants are enrolled, with 30 receiving placebo, 30 receiving vitamin D₂, and 30 receiving vitamin D₃. Total vitamin D intake is analyzed (primarily driven by dairy food intake and supplements), and patients with major absorptive disorders (intestinal bypass surgery, feeding tubes requiring liquid diets, etc) are excluded. Other factors that could affect vitamin D absorption such as alcohol intake are evaluated.

Co-administration of either vitamin D₂ or D₃ with atorvastatin results in an additional reduction on circulating cholesterol compared with atorvastatin and placebo.

### Example 3

A study is performed with 1000 IU/day of either vitamin D₂ or vitamin D₃ or identically packaged placebo for a period of 12 weeks. Cholesterol (total, LDL-cholesterol, HDL-cholesterol) and triglycerides are evaluated before and during supplemental vitamin D or placebo. Focus is on adult humans with or without known lipid disorders that are not receiving atorvastatin.

Data on one middle-aged female subject before and after 12 weeks of 1000 IU vitamin D (administered as single and combined forms of vitamin D₂ and D₃) revealed total and LDL-cholesterol were 241 and 144 mg/dL, respectively, before vitamin D and were 201 and 98 mg/dL, respectively, after about 12 weeks vitamin D supplementation. Data on one middle-aged male subject before and after 12 weeks of 1000 IU vitamin D revealed total and LDL-cholesterol were 238 and 125 mg/dL, respectively, before vitamin D and were 219 and 112 mg/dL, respectively after about 12 weeks vitamin D supplementation.

1. A method for lowering circulating LDL-cholesterol or total cholesterol in a human in need thereof, comprising: orally administering to the human a therapeutically effective amount of a statin and vitamin D daily for at least about 6 weeks,
wherein the human is selected from the group consisting of: a human unable to tolerate the statin when administered in a therapeutically effective dose and in the absence of vitamin D; a pre-menopausal female; a male without congestive heart failure; and a male with congestive heart failure; and
wherein the vitamin D is administered by one or more pharmaceutical compositions;
with the proviso that when the human is a male with congestive heart failure, the total daily amount of vitamin D administered by the one or more pharmaceutical compositions is equipotent to about 600 to about 1800 International Units (IU) of ergocalciferol or cholechalciferol.

2. The method of claim 1, wherein the human is unable to tolerate the statin when administered in a therapeutically effective dose and in the absence of vitamin D.

3. The method of claim 1, wherein the human is a pre-menopausal female.

4. The method of claim 3, wherein the human has congestive heart failure.

5. The method of claim 3, wherein the human does not have congestive heart failure.

6. The method of claim 1, wherein the human is a male without congestive heart failure.

7. The method of claim 1, wherein the human is a male with congestive heart failure.

8. The method of claim 1, wherein the human is selected from the group consisting of: a human unable to tolerate the statin when administered in a therapeutically effective dose and in the absence of vitamin D; a pre-menopausal female; and a male without congestive heart failure; and
the total daily amount of vitamin D administered by the one or more pharmaceutical compositions is equipotent to about 600 to about 2500 IU of ergocalciferol or cholechalciferol.

9. The method of claim 1, wherein the human is selected from the group consisting of: a human unable to tolerate the statin when administered in a therapeutically effective dose and in the absence of vitamin D; a pre-menopausal female; and a male without congestive heart failure; and
the total daily amount of vitamin D administered by the one or more pharmaceutical compositions is equipotent to about 1200 to about 2500 IU of ergocalciferol or cholechalciferol.

10. The method of claim 1, wherein the human is selected from the group consisting of: a human unable to tolerate the statin when administered in a therapeutically effective dose and in the absence of vitamin D; a pre-menopausal female; and a male without congestive heart failure; and
the total daily amount of vitamin D administered by the one or more pharmaceutical compositions is equipotent to about 600 to about 2000 IU of ergocalciferol or cholechalciferol.

11. The method of claim 1, wherein the total daily amount of vitamin D administered by the one or more pharmaceutical compositions is equipotent to about 600 to about 1800 IU of ergocalciferol or cholechalciferol.

12. The method of claim 1, wherein the total daily amount of vitamin D administered by the one or more pharmaceutical compositions is equipotent to about 600 to about 1200 IU of ergocalciferol or cholechalciferol.

13. The method of claim 1, wherein the total daily amount of vitamin D administered by the one or more pharmaceutical compositions is equipotent to about 800 to about 1000 IU of ergocalciferol or cholechalciferol.

14. The method of claim 1, wherein the vitamin D comprises one or more of: cholecalciferol, ergocalciferol, alfacalcidol, calcitriol, 22-oxacalcitriol, paricalcitol, doxercalciferol, and dihydrotachysterol₂.

15. The method of claim 1, wherein the vitamin D comprises ergocalciferol.

16. The method of claim 1, wherein the vitamin D comprises cholecalciferol.

17. The method of claim 1, wherein the statin is metabolized by cytochrome p450(CYP) enzyme (CYP3A).

18. The method of claim 1, wherein the statin is selected from the group consisting of: lovastatin, simvastatin, and atorvastatin.

19. The method of claim 1, wherein the statin is atorvastatin.

20. The method of claim 1, wherein the amount of statin required to achieve a particular decrease in LDL-cholesterol or total cholesterol is less than that required when the statin is administered in the absence of the one or more pharmaceutical compositions comprising vitamin D.

21. The method of claim 1, further comprising orally administering to the human calcium daily for at least about 6 weeks.

22. The method of claim 1, further comprising administering one or more additional therapeutic agents selected from the group consisting of: a non-statin lipid lowering agent, a HDL-raising agent, insulin, and a non-insulin diabetic agent.

23. The method of claim 22, wherein the one or more additional therapeutic agents are selected from the group consisting of: a bile acid sequestrant, a fibric acid derivative, an omega-3 fatty acid, niacin, a cholesterol absorption inhibitor, a cholesteryl ester transfer protein (CETP) inhibitor, insulin, a sulfonylurea, a biguanide, a meglitinide, a thiazolidinedione, a 6-alpha-glucosidase inhibitor, a glucagon-like peptide (GLP) analog, and a gastric inhibitory peptide (GIP) analog.

24. The method of claim 22, wherein the one or more additional therapeutic agents comprise ezetimide.

25. The method of claim 1, wherein the one or more pharmaceutical compositions comprises a pharmaceutical composition comprising vitamin D and calcium.

26. The method of claim 1, wherein the one or more pharmaceutical compositions comprises a pharmaceutical composition comprising a multivitamin composition comprising vitamin D.

27. The method of claim 1, wherein the human is at least about 12 years of age.

28. The method of claim 1, wherein the human does not have psoriasis.

29. The method of claim 1, wherein the human does not have osteoporosis.

30. The method of claim 1, wherein the human does not have multiple sclerosis.

31. The method of claim 1, wherein the human is diabetic.

32. The method of claim 1, wherein the human has not previously been treated with at least about 600 IU of ergocalciferol or cholecalciferol or equipotent amount thereof per day for at least about 6 weeks.

33. The method of claim 1, wherein prior to administration of the statin and vitamin D, the human has a combined 25-OH vitamin D₂ and 25-OH vitamin D₃ serum level of less than about 20 ng/ml, wherein the combined 25-OH vitamin D₂ and 25-OH vitamin D₃ serum level is measured by mass spectroscopy.

34. The method of claim 33, wherein after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy.

35. The method of claim 1, wherein prior to administration of the statin and vitamin D, the human has a combined 25-OH vitamin D₂ and 25-OH vitamin D₃ serum level of about 20 to about 30 ng/ml, wherein the combined 25-OH vitamin D₂ and 25-OH vitamin D₃ serum level is measured by mass spectroscopy.

36. The method of claim 35, wherein after at least about 6 weeks of the administration of the statin and vitamin D, the human has a combined 25-OH vitamin D₂ and 25-OH vitamin D₃ serum level of greater than about 30 ng/ml, wherein the combined 25-OH vitamin D₂ and 25-OH vitamin D₃ serum level is measured by mass spectroscopy.
